(19) United States
(12) Patent Application Publication
(10) Pub. No.: US 2025/0255744 A1
(43) Pub. Date: Aug. 14, 2025

(54) SYSTEMS AND METHODS FOR MODIFYING AN AIRWAY

(71) Applicant: The General Hospital Corporation, Boston, MA (US)

(72) Inventor: William G. Austen, Boston, MA (US)

(21) Appl. No.: 18/859,885

(22) PCT Filed: Apr. 25, 2023
(86) PCT No.: PCT/US2023/066180

§ 371 Date: Oct. 24, 2024

Related U.S. Application Data
(60) Provisional application No. 63/363,553, filed on Apr. 25, 2022.

(51) Int. Cl. A61F 5/08

(52) U.S. Cl. CPC A61F 5/08

(57) ABSTRACT

Systems and methods are provided that permanently modify an airway, such as a nasal airway without surgical intervention. The system to modify an airway can include one or more nasal inserts having different geometries or capable of being configured into different geometries to incrementally adjust bone or cartilage in the nasal airway over an extended period of time.

### CROSS-REFERENCES TO RELATED APPLICATIONS

This application is based on and claims priority to U.S. Provisional Patent Application No. 63/363,553 filed on Apr. 25, 2022, the entire contents of which is incorporated herein by reference.

### STATEMENT REGARDING FEDERALLY SPONSORED RESEARCH

Not Applicable.

### BACKGROUND

Airways, including nasal airways, can be obstructed due to a variety of factors and conditions. For example, an obstructed nasal airway can be caused by one or more of a deviated septum, enlarged turbinates, nasal polyps, enlarged adenoids, or a tumor. In general, obstructed airways can lead to breathing problems, including snoring, and increased nasal congestion. Some methods to alleviate airway obstruction include a surgical procedure to straighten bone and cartilage dividing the space between two nostrils (e.g., septoplasty). These procedures, however, can be painful, expensive, and introduce risk of surgical complications. Thus, it may be generally useful to modify an obstructed airway without surgical intervention.

### SUMMARY

In some aspects, the present disclosure provides systems and methods for modifying an airway through incremental adjustments over a treatment duration.

In another aspect, the present disclosure provides a nasal insert configured to be deployed over an extended period of into an airway of a person to modify a physical structure forming the airway. The nasal insert can include a nasal insert body having a size and shape matched to the airway of the person and having a corrective surface configured to engage one or more of nasal bone and cartilage surrounding the airway and to permanently adjust the structure of the airway and dimensioned to be inserted into a nasal passage of the person by the person to apply pressure via the corrective surface to the one or more of nasal bone and cartilage surrounding the airway. A passageway can be formed in the nasal insert body to allow air to enter and exit the airway when the nasal insert is inserted in the airway.

In some embodiments, a nasal insert can be manufactured using size and shape information of a person to match the airway of the person.

In some embodiments, a nasal insert can be manufactured using an additive manufacturing process using size and shape information acquired from medical images of a person.

In some embodiments, a nasal insert can be one in a set of nasal inserts, each nasal insert in the set of nasal inserts can have a different size or geometry. The set of nasal inserts can be configured to provide incremental adjustments to an airway over a treatment duration.

In some embodiments, a nasal insert can include a nasal body that includes a grip. The grip can be configured to facilitate insertion, removal, and adjustment of the nasal insert in an airway by a person.

In some embodiments, a nasal insert can include a grip. The grip can include an opening that is dimensioned to at least partially receive a digit of a person to rotate the nasal insert in an airway.

In some embodiments, a nasal insert can include a nasal insert body comprising hydrogel.

In some embodiments, a nasal insert can include a nasal insert body that is compressible.

In some embodiments, a nasal insert can include an adjustment system that incrementally changes a geometry of a nasal insert body to be matched to the size and shape of an airway.

In some embodiments, a nasal insert can include an adjustment system that includes threads or ribs.

In some embodiments, a nasal insert can include a nasal insert body. The nasal insert body can include one or more recesses or passages formed in a sidewall.

In another aspect, the present disclosure provides a nasal insert for modifying an airway of a person. The nasal insert can include a body sized for insertion into a nasal passage of a person and formed from a bio-compatible material. The body can include a corrective surface that is rigid or semi-rigid and configured to apply a corrective force to remodel an abnormality from the nasal passage during a treatment duration. An adjustment surface can be used to facilitate insertion, remove, and/or adjustment of the body within the nasal passage to apply a desired pressure on the abnormality over the treatment duration.

In some embodiments, a nasal insert can include an adjustment surface that includes a recess extending into the body and dimensioned to receive a digit of the person to facilitate self-deployment, self-adjustment, and/or self-removal.

In some embodiments, a nasal insert can include an adjustment surface that includes a sloped surface integrally formed with the body.

In another aspect, the present disclosure provides a method for using a nasal insert to modify an airway to correct an abnormality. The method can include identifying an abnormal airway, inserting a first nasal insert selected from a series of nasal inserts into the airway to engage a nasal passage surrounding the airway, the first nasal insert having a first geometry, removing the nasal insert after a first period of time configured to permanently remodel the nasal passage by a first increment, and inserting a second nasal insert selected from the series of nasal inserts into the nasal passage, the second nasal insert having a second geometry that is different from the first geometry and configured to permanently remodel the nasal passage by a second increment to correct the abnormality.

In some embodiments, a method for using a nasal insert to modify an airway to correct an abnormality can include removing a first nasal insert and adjusting the first nasal insert to form a second nasal insert.

In some embodiments, a method for using a nasal insert to modify an airway to correct an abnormality can include adjusting a first nasal insert which can include adjusting the orientation of a first corrective surface of the first nasal insert.

In some embodiments, a method for using a nasal insert to modify an airway to correct an abnormality can include using a series of nasal inserts that include at least five different nasal inserts.

In some embodiments, a method for using a nasal insert to modify an airway to correct an abnormality can include inserting a first nasal insert, which can include compressing a portion of the first nasal insert, moving the first nasal insert past a nostril opening, and expanding the first nasal insert within a nasal passage.

In another aspect, the present disclosure provides a method of producing a nasal insert for modifying an airway of a person. The method can include acquiring shape information about a nasal passage of the person to identify a nasal abnormality in the airway of the person, using the shape information, generating a nasal passage model, using the nasal passage model, determining a treatment plan to modify the nasal passage of the person to correct the nasal abnormality, and providing one or more nasal inserts having a geometry selected to implement the treatment plan to modify the nasal passage of the person to correct the nasal abnormality by deploying the one or more nasal inserts into the airway of the person.

In another aspect, the present disclosure provides a force applicator for modifying an airway. The force applicator and include a force applicator body dimensioned to apply pressure to one or more of a nasal bone and cartilage to permanently adjust the structure of the airway. A channel can be formed in the force applicator body to allow air to enter and exit the airway when the force applicator is inserted in the airway.

In some embodiments, a force applicator can be one force applicator in a series of force applicators. Each force applicator in the series of force applicators can have a different geometry. The series of force applicators can be configured to provide incremental adjustments to an airway over a treatment duration.

In another aspect, the present disclosure provides a method of using a force applicator to modify an airway. The method can include identifying an obstruction in the airway; inserting a first force applicator from a series of force applicators into a nasal passage, the first force applicator having a first geometry; removing the first force applicator after a first period of time; and inserting a second force applicator from the series of force applicators into the nasal passage, the second force applicator having a second geometry that is different from the first geometry.

In another aspect, the present disclosure provides a method of producing a force applicator for modifying an airway. The method can include scanning a nasal passage to identify a nasal obstruction and in airway; inputting patient data, including data collected during scanning, into a treatment algorithm; generating, via the treatment algorithm, an optimal nasal passage model; determining treatment duration; determining number of treatment increments; and fabricating one or more force applicators having different geometries, wherein the number of different geometries is the same number of treatment increments.

### BRIEF DESCRIPTION OF THE DRAWINGS

The invention will be better understood and features, aspects, and advantages other than those set forth above will become apparent when consideration is given to the following detailed description thereof. Such detailed description makes reference to the following drawings.

FIG. 1 is a partial cross-sectional front view of a nose having a deviated septum.

FIG. 2 is a partial cross-sectional front view of the nose of FIG. 1 including a nasal correction system according to one aspect of the present disclosure.

FIG. 3 is cross-sectional view of a nasal insert of the nasal correction system of FIG. 2.

FIG. 4 is a cross-sectional view of another nasal insert of the nasal correction system of FIG. 2.

FIG. 5 is a series of nasal inserts, shown in cross section, according to one aspect of the present disclosure.

FIG. 6 is an isometric view of a nasal insert according to one aspect of the present disclosure.

FIG. 7A is a side view of an adjustable nasal insert according to an aspect of the present disclosure.

FIG. 7B is a partial cross-sectional side view of the nasal insert of FIG. 7A in a first orientation.

FIG. 7C is a partial cross-sectional side view of the nasal insert of FIG. 7A in a second orientation.

FIG. 8 is a flowchart showing a method of generating a treatment plan to optimize a nasal passageway according to an aspect of the present disclosure.

FIG. 9 is a flowchart showing a method of using an airway modification system according to a treatment plan

### DETAILED DESCRIPTION OF THE INVENTION

The following discussion is presented to enable a person skilled in the art to make and use aspects of the present disclosure. Various modifications to the illustrated configurations will be readily apparent to those skilled in the art, and the generic principles herein can be applied to other configurations and applications without departing from aspects of the present disclosure. Thus, aspects of the present disclosure are not intended to be limited to configurations shown, but are to be accorded the widest scope consistent with the principles and features disclosed herein. The following detailed description is to be read with reference to the figures, in which like elements in different figures have like reference numerals. The figures, which are not necessarily to scale, depict selected configurations and are not intended to limit the scope of the present disclosure. Skilled artisans will recognize the non-limiting examples provided herein have many useful alternatives and fall within the scope of the present disclosure.

As described above, nasal airways can be obstructed due to a variety of factors and conditions. In some cases, surgical intervention (e.g., septoplasty) or temporary mechanisms may be used to alleviate or eliminate the airway obstruction. Surgical intervention can be painful, expensive, and introduce risk of complications. Additionally, surgical recovery can be lengthy and include residual pain, possibly extending through the nose, with distribution across cheeks, upper teeth, around the eyes, and in the forehead. Temporary mechanisms, such as certain devices that may be used to reduce snoring, for example, generally do not provide permanent or lasting results and only provide airway relief when the device is actively and properly in use.

Aspects of the present disclosure overcome these and other drawbacks of relieving obstructed airways. Aspects of the present disclosure can also be used to fix a deviated septum, aesthetically shape a patient's nose, or treat sinus disease. However, other applications are also possible.

The present disclosure provides a method of applying a relatively small yet consistent amount of pressure within a nasal cavity to reshape bones and/or cartilage over time, such that remodeling can be permanent. The pressure may be administered by a nasal insert that functions as a force applicator. One or more nasal inserts may be used to functionally yield a series of force applicators. In some embodiments, a nasal insert can be inserted into an airway to engage a nostril, bone, cartilage, or other structure for a period of time designed to have a therapeutic or remodeling effect. This insertion or deployment can be performed by the individual person without requiring a trained clinician or other assistant. After a first period of time, a modified or second nasal insert can be deployed by the individual to achieve a second force applicator having a different geometry from the first nasal insert to adjust the pressure applied within the nose.

Over time, an incremental increase and/or adjustment of pressure applied by a nasal insert, a series of nasal inserts, or a plurality of series of nasal inserts can modify structures surrounding the airway. The result may therapeutic, such as to provide permanent nasal passageway relief without surgical intervention, or aesthetic. In general, in some embodiments, the amount of pressure applied by the nasal insert is designed to facilitate extended use and, thereby, not provide extreme discomfort to the person such that they are unable to sleep or be inhibited from accomplishing and participating in daily activities. To this end, as will be described, the nasal insert may be designed to fit entirely within the nasal passage and may be formed of transparent or other materials, such that the nasal insert is not observed by others when deployed into the airway/nostril.

In general, the treatment duration and number of increments of adjustment (e.g., the number of nasal inserts in a series of nasal inserts or range of adjustability of a given nasal insert) may be unique to each patient. In some embodiments, to initiate treatment, a patient may undergo measurements or one or more scans or molding processes. A medical professional, specialist, or technician may then determine treatment duration and anticipated number of incremental force applicators needed successfully modify an airway. In one example, a patient may wish to reduce or eliminate snoring by modifying their nasal airway. The patient can then have an image or mold acquired to determine size and/or geometric information about their nasal passage and airway. A specialist or computer system can determine a treatment plan using this information. An example treatment plan can include a duration of one year with a nasal insert series and can include, for example, multiple nasal inserts, each designed to apply incremental pressure within the patient's nasal passageway for the treatment duration.

Other treatment durations and number of nasal inserts in a series may be desirable, and may depend on the age, gender, bone structure, severity of nasal abnormality or passageway obstruction, and other patient-specific factors. Treatments to modify a nasal structure according to embodiments of the present disclosure can also differ in the length of time that each nasal insert is in the nasal passageway. For example, while a treatment duration might be one year, each applicator may spend 6-10 hours per day in the nasal passageway. Additionally, in some embodiments, within one treatment plan, the time each nasal insert spends in a nasal passageway may vary depending on the stage of treatment. It should be understood that these lengths of time (both treatment duration and time each nasal insert spends in a nasal passageway) are by way of example and may be varied by minutes, hours, days, or years on a patient to patient basis.

Some treatment plans may include scanning or otherwise observing a patient's abnormal nasal passage and prescribing a first series of nasal inserts having a plurality of differing nasal inserts. In some embodiments, nasal inserts within a series (or between series) can differ by one or more of geometry, texture, or material. Once the patient progresses through the first series of nasal inserts, they may return to their treatment provider or otherwise provide a follow-up scan of their nose in order to assess treatment progression. The patient may then be provided a second series of nasal inserts having a plurality of differing nasal inserts. The process of assessing treatment progression and employing additional series of nasal inserts may continue until the nasal abnormality is sufficiently corrected. In general, sufficiently corrected may include an optimal nasal passageway, an improved nasal passageway, or an altered nasal passageway that achieves a particular goal (e.g., to reduce or eliminate snoring).

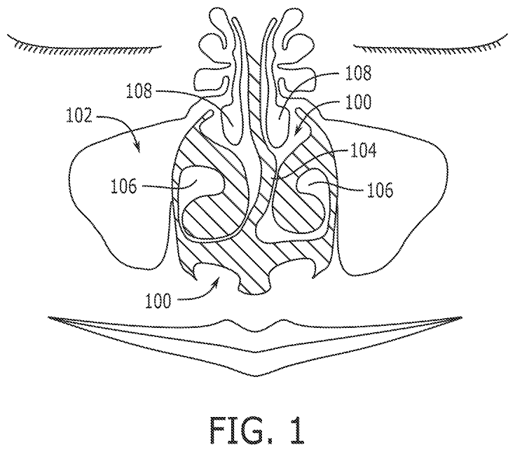

As described above, a variety of conditions can cause an obstruction or general abnormality in a nasal passage that a patient may desire (or need) to have fixed. For example, a deviated septum is a condition in which the bone and cartilage that divide the nasal cavity of the nose in half (i.e., the nasal septum) is significantly off center or crooked. In general, a deviated septum can make breathing difficult and/or provide unwanted cosmetic abnormalities. In this regard, FIG. 1 illustrates one example of a nasal passage 100 of a nose 102 having a deviated septum 104.

As further illustrated in FIG. 1, nasal passages generally include a plurality of turbinates. Turbinates can include bony structures inside a nose, covered by soft tissue. They can regulate airflow and warm and humidify air that is inhaled. As shown in FIG. 1, the nasal passage 100 includes a pair of inferior turbinates 106 and a pair of middle turbinates 108. In general, allergies or other illnesses can irritate a turbinate and cause it to swell or enlarge. Such swelling can reduce breathing ability through the nasal passage. Embodiments of the invention can reduce, relieve, or alter these and other nasal abnormalities and conditions.

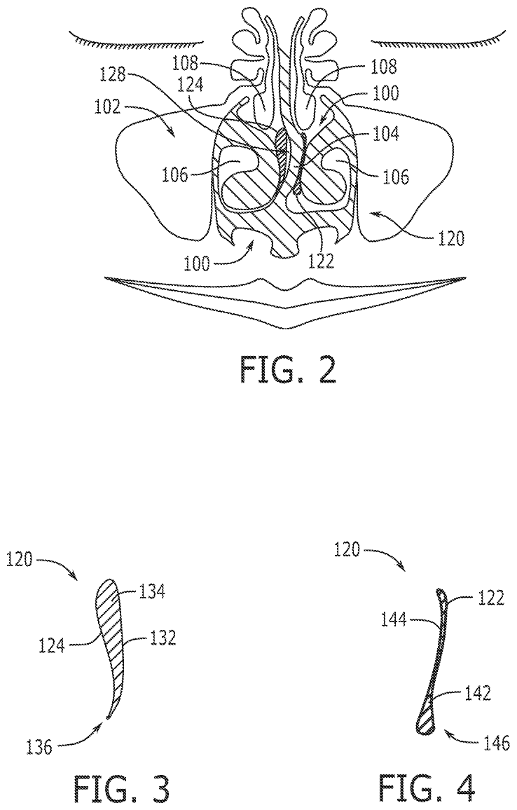

FIG. 2 illustrates the nasal passage 100 including one example of a nasal correction system 120. In the illustrated embodiment, the nasal correction system 120 includes a nasal insert 122 positioned at a convex side of the deviated septum 104 and a nasal insert 124 positioned at a concave side of the deviated septum 104. The nasal insert 122 can be configured as a force applicator that can apply a corrective force on the deviated septum 104. The corrective force may be small enough that the patient can tolerate wearing the nasal insert 122 for hours, days, weeks, etc. at a time. The concept of applying a corrective force may be, in some instances, similar to that of braces: applying a corrective force strong enough to move a structure over time, while being weak enough not to cause intolerable pain or discomfort.

Congruent with the nasal insert 122, the nasal insert 124 can be configured as a counter pressure insert. For example, the nasal insert 124 on the concave side of the deviated septum 104 may apply an opposing force to the nasal insert 122 on the convex side of the deviated septum 104. The opposing force applied by the counter pressure insert (i.e., the nasal insert 124) may be configured to prevent or reduce an overcorrection of the deviated septum 104. Additionally, the counter pressure insert 124 can be used to prevent movement of the septum in the non-deviated areas (e.g., the top and bottom portion of the septum in FIG. 2). In the embodiment shown, the nasal insert 124 may form a gap 128 with the septum 104. The gap 128 may provide a space for the septum 104 to move into as the force applicator (i.e., the nasal insert 122) urges the septum 104 to straighten out. During a treatment duration, as the gap 128 closes, the septum 104 is moved into a corrected (i.e., straightened) orientation, and the nasal insert 124 can prevent the septum 104 from deviating in another, unwanted direction.

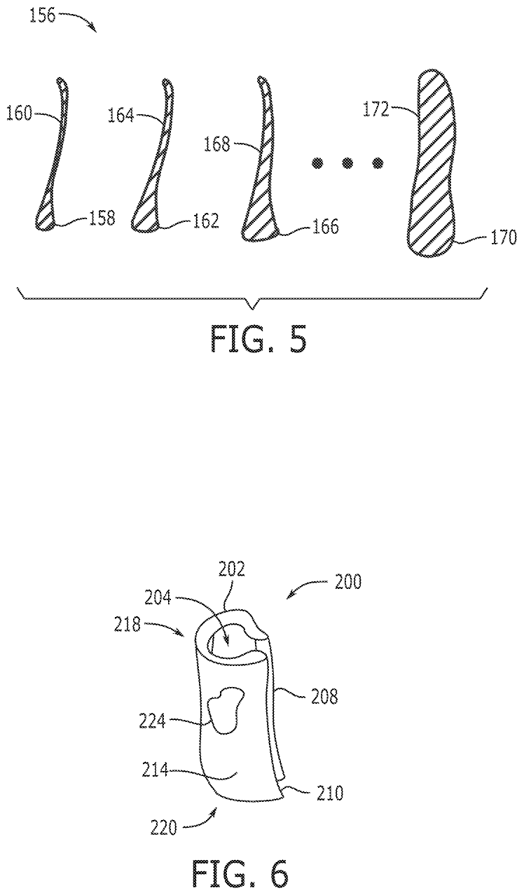

In use, the nasal correction system 120 can include a plurality of nasal inserts, including the nasal inserts 122 and 124. The plurality of nasal inserts can be part of a set of nasal inserts (see, for example, FIG. 5) that a patient will progress through during a treatment duration. For example, a counter pressure insert (e.g., the nasal insert 124) may be inserted on one side of a septum, and a force applicator (e.g., the nasal insert 122) may be inserted on the other side of the septum for a first duration (e.g., 1 week). After the first duration, the patient may remove the force applicator (e.g., the nasal insert 122) and replace it with a second nasal insert in the set of nasal inserts. In some uses, the patient may keep the counter pressure insert in the nasal passage during the duration of the treatment. However, in other instances, the patient may remove, discard, and/or replace the counter pressure insert.

In some instances, treatment may be revised or completed after the patient has progressed through each subsequent nasal insert in a set of nasal inserts. For example, after completing a first set of nasal inserts, the patient's nasal passage may be evaluated to determine if additional nasal inserts are required to continue to correct the nasal abnormality. In another example, the treatment may be completed after the patient has progressed through a plurality of sets of nasal inserts and their nasal defect has been corrected, eliminated, or reduced. For example, the treatment to fix a deviated septum may be complete when the septum is substantially straight, breathing has improved, and/or snoring has been reduced or eliminated.

FIGS. 3 and 4 illustrate the nasal inserts 124, 122, respectively, of the nasal correction system 120 of FIG. 2. As shown in FIG. 3, the nasal insert 124 can include a body 132 having a corrective surface 134. The corrective surface 134 can provide an overcorrection barrier during the treatment process so that, for example, a deviated septum is not urged too far in an opposing direction and the septum can be aligned with the corrective surface 134. In this regard, the corrective surface 134 may be substantially parallel (i.e., deviating from parallel by 5 degrees) to a normal or corrected septum.

The nasal insert 124 can further include a grip portion 136 configured to facilitate insertion, removal, and/or adjustment of the nasal insert 124. In some embodiments, the grip portion 136 can include an opening or recess dimensioned to at least partially receive a digit to help a patient rotate or remove the nasal insert 124 within the nasal passage. In other embodiments, the grip portion 136 can include one or more of a ledge, a tapered edge, or a cutout to aid gripping and adjusting the nasal insert 124. Additionally, in some embodiments, one or more surfaces of the body 132 can include a hydrogel material to provide both comfort and rigidity for the patient.

FIG. 4 illustrates the nasal insert 122, which includes a body 142 having a corrective surface 144. In the illustrated embodiment, the corrective surface 144 includes a curved surface; however, the corrective surface of a force applicator nasal insert may progressively change between inserts as the nasal abnormality is corrected. In the illustrated embodiment, the corrective surface 144 defines a geometry similar to that of the deviated septum 104 of FIG. 2 with a slightly less deformation so that the corrective surface 144 can apply a corrective force on the deviated septum 104.

Similar to the nasal insert 124, the nasal insert 122 of FIG. 4 can further include a grip portion 146 configured to facilitate insertion, removal, and/or adjustment of the nasal insert 122. In some embodiments, the grip portion 146 can include an opening or recess dimensioned to at least partially receive a digit to help a patient rotate or remove the nasal insert 122 within the nasal passage. In other embodiments, the grip portion 146 can include one or more of a ledge, tapered edge, or a cutout to aid gripping and adjusting the nasal insert 122. Additionally, in some embodiments, one or more surfaces of the body 142 can include a hydrogel material to provide both comfort and rigidity for the patient.

In general, nasal inserts described herein can include a corrective surface that is configured to abut a septum (e.g., a deviated septum), or other abnormality or obstruction in the nasal passage to apply a corrective force over a treatment period to deliberately alter the abnormality to improve breathing or aesthetics over time. The body of a nasal insert can further include a secondary corrective surface, which may be opposite a main corrective surface (the corrective surface that abuts the septum or obstruction). The secondary corrective surface can be configured to engage other structures within the nasal passage so that the nasal insert is generally wedged between such other structure and the obstruction.

As briefly described above, the nasal correction system 120 shown in FIGS. 2-4 can include the nasal inserts 122, 124, among other nasal inserts. Alternatively, a nasal correction system may include one or more nasal inserts that are configured to only be inserted into a single nasal passage. It should be appreciated that the type of correction needed, and other patient-specific factors, can influence whether one or more nasal inserts are inserted on one more sides of a septum throughout (including intermittent periods) of the treatment.

With reference now to FIG. 5, as described above, a nasal insert according to embodiments of the present disclosure may be part of a set or series of nasal inserts, such as the set 156. As illustrated by way of example, the first nasal insert 158 in the set 156 has the same geometry as the nasal insert 122 of FIG. 4 and includes a corrective surface 160; however, other geometries are possible. The second nasal insert 162 of the set 156 defines a second geometry that is different than the geometry of the first nasal insert 158. The second nasal insert 162 similarly includes a corrective surface 164. As shown, the corrective surface 164 of the second nasal insert 162 defines a lesser curvature than the corrective surface 160 of the first nasal insert 158.

With continued reference to FIG. 5, the third nasal insert 166 of the set 156 defines a third geometry that is different than the geometry of the first nasal insert 158 and the second nasal insert 162. The third nasal insert 166 can include a corrective surface 168. As shown the corrective surface 168 of the third nasal insert 166 defines a lesser curvature than the corrective surface 164 of the second nasal insert 162, and thus a lesser curvature than the corrective surface 160 of the first nasal insert 158. As shown, the set 156 can include n nasal inserts (n being any positive integer). The n^{th} nasal insert 170 can include a corrective surface 172 that defines a curvature less than the (n-1)^{th} nasal insert, however, other variations in geometry are also possible and patient-dependent.

In general, the number (n) of nasal inserts in a set may be determined by the severity of a deviated septum or other abnormality, along with other patient-specific characteristics. Additionally, the number (j) of sets of nasal inserts may be determined by the severity of a deviated septum or other abnormality, along with other patient-specific characteristics and progress during treatment. For example, in use, a patient may return for additional scans or evaluations before receiving second, third, fourth, etc. sets of nasal inserts. In general, treatment for permanently fixing or adjusting a nasal abnormality may be complete after the removal of the n^{th} nasal insert from the j^{th} set of nasal inserts.

Additionally, while the set 156 of nasal inserts shown in FIG. 5 indicates lessening curvatures of corrective surfaces during the progression of the nasal inserts in the set 156, other progressive geometries are possible. For example, the geometries of the corrective surfaces or overall bodies of the nasal inserts may increase in curvature, decrease in curvature, or otherwise change. The progressive geometric changes of the nasal inserts within a set of nasal inserts can provide a continuing necessary corrective force to permanently adjust a nasal abnormality over time.

In general, the inserts described herein may be irregular in shape and reflect a lack of symmetry. The insert may be irregular or asymmetrical because the shape of the insert is designed specifically to the patient and the patient's anatomy. The lack of symmetry may include one or both of vertical asymmetry or horizontal asymmetry. This is because the insert is designed for the patient's irregular and correcting anatomy, rather than an abstract assumption of anatomy or structure. Furthermore, the insert may prioritize both the patient's specific anatomy and the functional outcomes being sought with the therapeutic insert. For example, in addition to the current anatomy and the desired remodeling to be achieved by the insert, the design of the insert may consider intermediate or ultimate functionality and/or the influence of the insert on adjacent structures and the functionality of those adjacent structures.

FIG. 6 illustrates another example of a nasal insert 200 that can be used to non-surgically modify a patient airway. The nasal insert 200 may be configured as a force applicator and includes a body 202 that defines a passageway 204. In general, a passageway in a nasal insert can allow air to move through the nasal insert so that, when inserted correctly into a nasal passageway, a patient's breathing is not impaired. This, coupled with other mechanisms that reduce discomfort, can promote extended use of nasal insert described herein.

As shown in FIG. 6, the nasal insert 200 can generally form a cylinder. Additionally or alternatively, the nasal insert 200 can be shaped to match a nostril shape of a patient. The passageway 204 can be fully or partially formed by the body 202. For example, in the illustrated embodiment, the passageway 204 is partially formed by the body 202, which defines a C-shaped cross section. In other embodiments, the passageway 204 may be fully formed by the body 202 so that the cross-section is circular (or oval-shaped, or irregular). In the illustrated embodiment, the C-shaped cross section can facilitate insertion of the nasal insert 200 into a nasal cavity. For example, first and second ends 208, 210 of the body 202 can be pinched together (i.e., to close the C-shaped cross section) to reduce a width of the nasal insert 200 during insertion. As the body 202 is released (e.g., from the fingertips of a user), the nasal insert 200 can expand within the nasal passage to provide a corrective force to a targeted abnormality or obstruction.

In use, a corrective surface 214 can be used to apply a pressure on the abnormal structure in the nasal passageway. For example, the corrective surface 214 illustrated in FIG. 6 can be used to apply pressure to a septum to correct a deviated septum. Additionally, in the illustrated embodiment, the corrective surface 214 can extend axially between a first end 218 and a second end 220 of the body 202. In other embodiments, a corrective surface may only extend partially between a first end and a second end of the body. Additionally or alternatively, in some configurations, a plurality of corrective surfaces can be disposed about a body of a nasal insert so that one or more corrective surfaces apply an appropriate corrective force. In general, an appropriate corrective force can be a deliberate force applied to a nasal passage to correct an abnormality, defect, injury, etc., such as a deviated septum or other nasal bone or cartilage irregularity. The “abnormality” may also be a structure desired to be changed for aesthetic purpose. As described above, such irregularity may be slowly corrected over time via continuous or intermittent compression, tension, or otherwise intentional force applied to one or more area of a nose.

In one example of use, the nasal insert 200 can be inserted into a nasal passage so that the first end 218 enters the nasal passage first. The corrective surface 214 can be, for example, aligned with a septum so that at least a portion of the septum is flush with the corrective surface 214. In some configurations, the corrective surface 214 may include a contoured surface that is similar to that of a septum. Once inserted, the user may resume normal breathing through their nose, and the passageway 204 can allow air to pass freely through the nasal insert 200 between the user and the ambient environment.

With continued reference to FIG. 6, the body 202 can include one or more surface deviations 224. In general, a surface deviation can include a recess, a protrusion, a channel, a hole, a slit, or any element of the body of the nasal insert 200. In the illustrated example of FIG. 6, the surface deviations 224 include recesses that extend into the body 202 at the surface. The recesses 224 can extend through the thickness of the surface so that there is an intersection between the passageway 204 and the recess 224. Thus, the surface area of the nasal insert 200 in contact with the nasal wall or other structure is reduced when it is deployed by a person and can be minimized to only surfaces desired or required for achieving the permanent remodeling or therapeutic effect.

In some configurations, one or more recesses or surface deviations 224 can promote the flexibility of the body 202 to facilitate inserting the nasal insert 200 into a nasal passage. In particular, an opening to a nasal passage may have a smaller area than inside the nasal passage adjacent to the nostril sill. Therefore, it may be advantageous to flex (e.g., compress) at least a portion of the body 202 so that the nasal insert 200 can be easily inserted. In other configurations, one or more recesses 224 can improve airflow through the nasal insert 200 to promote unrestricted breathing or maintain senses such as smelling or functions such as mucus secretion. Additionally or alternatively, one or more recesses 224 can reduce the amount of contact area between the nasal insert 200 and the internal walls of the nasal passage to reduce discomfort, damage, or inflammation of the nasal passage.

As described above, configurations of the disclosure provide a method of applying pressure within a nasal cavity to reshape bones and/or cartilage over time. The pressure can be applied via varying or incrementally changing nasal inserts that are configured to fix nasal abnormalities at a comfortable and user-friendly way. In this regard, it should be understood that any of the nasal inserts described herein (above and below) may be one example of a nasal insert from a set of nasal inserts and that subsequent nasal inserts may have slightly different geometries, curvatures, or corrective surface features that are configured to progress the reshaping of nasal bone and/or cartilage.

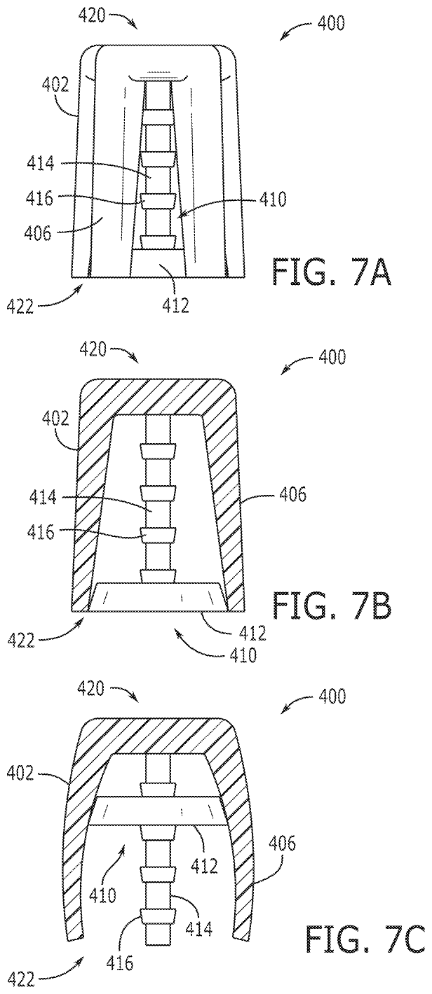

FIGS. 7A-C illustrate another example of a nasal insert 400 according to the present disclosure. The nasal insert 400 includes a body 402 that can define a passageway (not shown) extending therethrough. As discussed above, a passageway can allow air to move through the nasal insert so that when inserted correctly into a nasal passageway, a patient's breathing is not impaired or uncomfortably restricted. The body 402 can also include a corrective surface 406 configured to apply a corrective force within a nasal passage. As described above, the corrective force applied by a nasal insert may be adjusted over a treatment period to slowly and methodically correct nasal abnormalities.

In the illustrated configuration, the corrective surface 406 may be changeable via an adjustment system 410. The adjustment system 410 can include an adjuster 412. The adjuster 412 can be configured as a nut, ring, or other moveable component that can be secured relative to the body 402 to provide adjustments (e.g., incremental adjustments) of one or more corrective surfaces 406. As show in FIGS. 7B and 7C, the adjuster 412 is configured to move axially along a central structure 414 having a plurality of incremental adjustment ribs 416. In some configurations, the central structure 414 may be hollow and include a passageway extending therethrough. In use, as the adjuster 412 is moved and secured at each incremental adjustment rib 416, the corrective surfaces 406 are incrementally changed. The incremental change of the corrective surfaces 406 can apply varying forces (i.e., varying in magnitude and/or position) to a portion of a nasal passage. In other embodiments, the nasal insert 400 may be infinitely adjustable.

In some configurations, the incremental adjustment ribs 416 may provide sufficient resistance such that the adjustment system 410 is not inadvertently adjusted when the nasal insert 400 is correctly positioned in the nasal cavity. In other configurations, an adjustment system can include one or more adjustment mechanisms including threads, snaps, adessive, and telescoping components, for example, to adjust the geometry of the nasal insert incrementally during the treatment duration.

As briefly described above, a variety of manufacturing methods can be used during the production of a nasal insert according to the present disclosure. For example, the corrective surfaces 406 may be integrally formed with the body 402 and extend from a first end 420 toward a second end 422 of the nasal insert 400. The adjustment system 410 can then be secured at the first end 420 via the central structure 414. In other configurations, the central structure 414 may be integrally formed with the body 402.

In use, the nasal insert 400 may be positioned in a first incremental position of n positions (n being the number of increments available on a single nasal insert) via the adjustment system 410 and inserted into a nasal passage. The nasal insert 400 may be used by first inserting a first end 420 into the nostril, and then, in some configurations, moving the nasal insert 400 generally upward until a second end 422 of the nasal insert 400 is received within the nostril. After a first prescribed amount of time, the nasal insert 400 may be removed from the nostril. positioned in a second incremental position of n positions, and reinserted into the nostril. Contrastingly, in other configurations, the nasal insert 400 may be incrementally adjusted after a first prescribed amount of time while the nasal insert 400 remains in the nasal passage. Within a treatment plan, a series, array, or otherwise kit of nasal inserts similar to the nasal insert 400 may be used. In other embodiments, the force applied within a nasal passage may be progressively adjusted by repositioning a nasal insert within the nasal passage. For example, rotating a nasal insert within a nasal passage may provide varying corrective force used to move or correct a defect.

As described above, configurations of the present disclosure provide a method to modify a nasal airway by applying a small amount of pressure within a nasal cavity to reshape bones and/or cartilage overtime. As the bones or cartilage are moved within the nose, adjustments (e.g., incremental adjustments) can be made to a nasal insert so that an updated amount of pressure or position of pressure can be used to continue correcting obstructions or deviations in the nasal passage. In some configurations, the incremental adjustments can be made using a series of nasal inserts each having slightly different geometries. In other configurations, the incremental adjustments can be made using one or more of an adjustable nasal insert, such as the nasal insert 400 for example.

In one example, a medical professional or specialist may prescribe a treatment plan to modify an airway (e.g., fix a deviated septum). The treatment plan can be a predetermined length of time (e.g., 18 months) and can include the nasal insert 400. At the beginning of the treatment plan, the nasal insert 400 may be adjusted to a first position via the adjustment system 410. After the patient uses the nasal insert 400 for a certain amount of time in the first position (e.g., 8 hours per day for 4 days), the patient (or other user) may adjust the nasal insert 400 to a second position via the adjustment system 410. In some configurations, the nasal insert 400 may have a plurality of incremental, adjustable positions (e.g., 10 positions). In some configurations, once a first nasal insert 400 has gone through each of the adjustable positions during a first time segment of the treatment duration, a second nasal insert similar to the nasal insert 400 can be used to continue the airway modification process. In general, if a nasal insert includes an adjustment system, then there may be fewer nasal inserts in a set of nasal inserts compared to nasal inserts without an adjustment system.

In general, each of the nasal inserts described above can include a compressible or semi-rigid structure and material that can allow the nasal insert to be at least slightly deformed during insertion, but have sufficient structure or semi-rigidity to apply a corrective force in the nasal passageway. The nasal inserts can include one or more passageways and/or channels that can allow air to move through the patient's nasal airway. The passageways can be formed in the body of the nasal inserts, and can also include openings formed in mesh, webbing, or netting of the nasal insert. However, in other embodiments, the nasal insert can be a solid body.

In some exemplary configurations, nasal inserts according to embodiments of the invention can include expandable, inflatable, and/or absorbable materials. For example, a nasal insert can include an absorbable material so that before the nasal insert is inserted into the nasal passageway, the nasal insert defines a first geometry, and after the nasal insert has been inserted into the nasal passageway, the nasal insert defines a second geometry that is configured to apply a corrective force to a nasal abnormality. The second geometry can be a result of the nasal insert absorbing and expanding/deforming. In some embodiments, the nasal insert may be configured to expand when introduced to a fluid, such as mucus.

In other embodiments, a nasal insert can be configured as a cage. The cage can have wire-like or mesh structures that can provide corrective forces or counter pressures within a nasal passageway. In some examples, the cage can be deployed into a nasal passageway, and then manipulated by an outside force (e.g., finger, forceps, or other tools) to the appropriate geometry to begin to correct a nasal abnormality. During the treatment duration, the cage can be manipulated or adjusted to provide continued therapeutic or remodeling effects.

Further, in some embodiments, the rigid structure can include a laminate structure having one or more layers fixed together to form a hard material. In general, the hardness of the material allows the nasal insert to apply a corrective force within a nasal cavity while providing a degree of softness that is comfortable in the nasal passage. In some configurations, a degree of softness of a nasal insert can be achieved via material selection. In general, the material of the nasal insert can be bio-compatible.

For example, certain polymers, such as hydrogel, can be used to form a relatively rigid structure capable of applying pressure when inserted into a nasal passage while also providing soft surfaces and edges to avoid scraping, cutting, or otherwise hurting the patient. As noted above, a variety of materials, including combinations of materials, can be used in a nasal correction system. Such materials can include, for example, polymers (e.g., plastics), metal, synthetic fibers, and other composites.

In some examples, one or more components of the nasal correction system (e.g., one or more force applicators or counter pressure inserts) can include an alloy, such as nickel titanium (e.g., nitinol). Nitinol, and other shapable alloys and composites may include advantageous material characteristics, including a shape memory effect (e.g., a material that can be deformed when cold but returns to its pre-deformed shape when heated). In some embodiments, shapable materials can be reshaped or adjusted during a treatment process so that a single nasal insert may be used to apply a variety of corrective forces.

Other bio-compatible materials can include, for example, silicone, alumina, bioglass, cobalt-chromium, hydroxyapatite (HA), polyvinylchloride (PVC), polypropylene (PP), polytetrafluoroethylene (PTFE), polymethylmethacrylate (PMMA), polyethylene (PE) polysulfone (PS), polyurethane (PU), polyetheretherketone (PEEK) polycarbonate, polyetherimide (PEI) poltrimethylcarbonate, stainless steel, titanium and titanium alloys, and zirconia.

Additionally, each of the nasal inserts described above can be produced via a variety of manufacturing techniques, including printing, molding, overmolding, casting, etc. In some configurations, it may be beneficial (e.g., cost-effective) to produce a series of nasal inserts via additive manufacturing (i.e., 3D printing) given the variation of shapes and sizes of nasal inserts across different patients and even within single treatment plans.

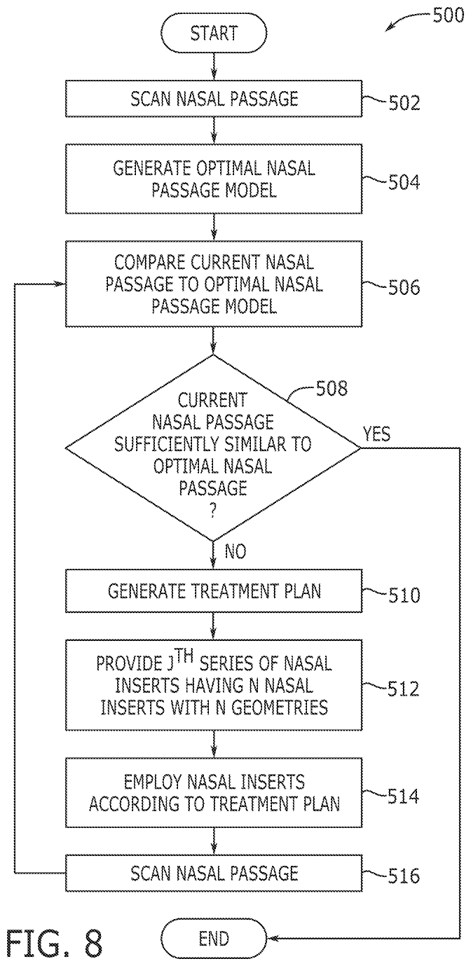

Referring now to FIG. 8, configurations of the present disclosure provide a method of generating a treatment plan to modify and/or optimize a nasal passageway. The method 500 employs nasal inserts to adjust the nasal passageway. The nasal inserts may be of a variety of nasal inserts, including the nasal inserts described above. The method 500 can be implemented when a medical professional or specialist determines that a patient has a nasal obstruction or abnormality and would benefit from modifying their airway.

The method illustrated in FIG. 8 describes scanning a nasal passage at step 502. At step 504, an optimal nasal passage model may be generated, such as by a program or a technician, for example. The optimal nasal passage may be a general optimal nasal passage or may be specific to the patient and based on the patient's nasal scan. In general, the optimal nasal passage model is a model of what the patient's nasal passageway may be after modification via treatments described herein. At step 506, the current nasal passage may be compared to the optimal nasal passage to evaluate treatment options.

If the current nasal passage is not sufficiently similar to the optimal nasal passage, and/or the patient is experiencing breathing difficulty, snoring, or other unwanted symptoms of a nasal abnormality, a treatment plan may be created. Otherwise, if the patient's current nasal passage is sufficiently similar to the optimal nasal passage such that they are not experiencing breathing difficulty, snoring, or other unwanted symptoms of a nasal abnormality, no further treatment is necessary, as indicated at decision 508.

If airway modification is necessary because the current nasal passage is not sufficiently similar to the optimal nasal passage, a treatment plan can be generated at step 510. In one configuration, the treatment plan can be generated with the help of an algorithm capable of receiving scans or measurements and generating a treatment duration and an appropriate number of incremental steps for correctly and permanently modifying the airway.

In other embodiments, the treatment plan can include determining how many nasal inserts should be included in a set of nasal inserts for a first round (or subsequent round) of treatment. For example, a specialist or a computer program may indicate that ten nasal inserts should be included in a first set of nasal inserts before further evaluation is necessary. After generating the treatment plan, the patient may be given a set of nasal inserts at step 512. The number of sets of nasal inserts required during the entire treatment can vary on a patient to patient basis. As a result, the number of sets in a treatment is designated as j. Similarly, the number of nasal inserts in a set can vary on a patient to patient basis. As a result, the number of nasal inserts in a set is designated as n.

Step 514 includes employing n nasal inserts in the j^{th} set of nasal inserts of a prescribed duration. For example, if the first set of nasal inserts includes ten nasal inserts, and each insert is prescribed to be inserted for one week at a time each, then step 514 may last 10 weeks. After the progression of the nasal inserts in the current (j) set is complete, the patient may return to their provider for an evaluation (e.g., a scan) on the modification process at step 516. Such evaluation (e.g., scan) can then be used to again, at step 506, compare the current nasal passage to the optimal nasal passage. If the nasal passage has been sufficiently modified, then treatment may be complete. In some instances of extreme deformation or abnormalities, a more permanent retainer nasal insert may also be used to prevent the obstruction from returning after the initial treatment period.

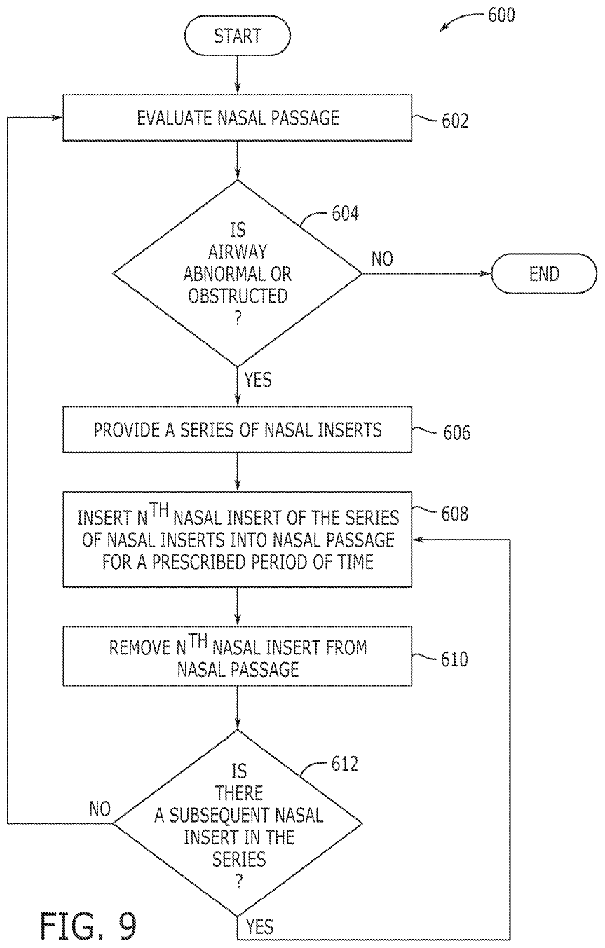

Referring now to FIG. 9, the method 600 provides a method of using an airway modification system according to a treatment plan. The method 600 describes using nasal inserts, such as the nasal inserts described herein. The method 600 includes, at step 602, evaluating a nasal passage to identify and/or quantify a nasal abnormality. If an abnormality or obstruction is detected at decision 604, then a series of nasal inserts may be provided to the patient at step 606. At step 608, the patient may insert (or have inserted) one of the nasal inserts of n inserts in the series. As shown in step 610, the patient can remove the nasal insert after the prescribed amount of time has passed.

In general, the series of nasal inserts would be used to incrementally or continuously modify the airway. After the insertion and removal of the first nasal insert, it is likely that the airway would still be obstructed or disfigured and the patient should progress to a next nasal insert in the series. Therefore, at decision 612, the patient would return to step 608 and insert the second (or n^{th}) insert into their nasal passage. In some embodiments where the nasal insert is adjustable, returning to step 608 may include adjusting the nasal insert into an n^{th} position or orientation. The cycle of inserting the nth nasal insert and removing the n^{th} nasal insert may continue until the patient has progressed through all n inserts in the series.

At decision 612, if there are no subsequent nasal inserts in the series, then the patient may return to their provider for a revaluation of their nasal passage at step 602. After the evaluation, if there is still and obstruction (e.g., still a deviated septum), then the patient may return to step 606 and proceed with a next series of nasal insert. If the obstruction or deviation is cured, then the treatment may be complete.

It should be appreciated that the length of time that a nasal insert is positioned within a nasal passageway (see step 608) may not be a consecutive amount of time. For example, a treatment plan may require the patient to wear a single nasal insert for a cumulative of 24 hours, which may be spread across multiple days or time increments. Thus, step 610 may refer to removing the n^{th} nasal insert after the entirety of the prescribed amount of time for that insert has elapsed.

Within this specification embodiments have been described in a way which enables a clear and concise specification to be written, but it is intended and will be appreciated that embodiments may be variously combined or separated without parting from the invention. For example, it will be appreciated that all preferred features described herein are applicable to all aspects of the invention described herein.

Thus, while the invention has been described in connection with particular embodiments and examples, the invention is not necessarily so limited, and that numerous other embodiments, examples, uses, modifications and departures from the embodiments, examples and uses are intended to be encompassed by the claims attached hereto. The entire disclosure of each patent and publication cited herein is incorporated by reference, as if each such patent or publication were individually incorporated by reference herein.

Various features and advantages of the invention are set forth in the following claims.

We claim:

1. A nasal insert configured to be deployed over an extended period into an airway of a person to modify a physical structure forming the airway, the nasal insert comprising:
a nasal insert body having a size and shape matched to the airway of the person and having a corrective surface configured to engage one or more of nasal bone and cartilage surrounding the airway and to permanently adjust the structure of the airway and dimensioned to be inserted into a nasal passage of the person by the person to apply pressure via the corrective surface to the one or more of nasal bone and cartilage surrounding the airway; and
a passageway formed in the nasal insert body to allow air to enter and exit the airway when the nasal insert is inserted in the airway.

2. The nasal insert of claim 1, wherein the nasal insert is manufactured using size and shape information of the person to match the airway of the person.

3. The nasal insert of claim 2, where the nasal insert is manufactured using an additive manufacturing process using size and shape information acquired from medical images of the person.

4. The nasal insert of claim 1, wherein the nasal insert forms one in a set of nasal inserts, each nasal insert in the set of nasal inserts having a different size or geometry, and
wherein the set of nasal inserts are configured to provide incremental adjustments to the airway over a treatment duration.

5. The nasal insert of claim 1, wherein the nasal insert body includes a grip configured to facilitate insertion, removal, and adjustment of the nasal insert in the airway by the person.

6. The nasal insert of claim 5, wherein the grip includes an opening dimensioned to at least partially receive a digit of the person to rotate the nasal insert in the airway.

7. The nasal insert of claim 1, wherein the nasal insert body comprises hydrogel.

8. The nasal insert of claim 1, wherein the nasal insert body is compressible.

9. The nasal insert of claim 1, further comprising an adjustment system that incrementally changes a geometry of the nasal insert body to be matched to the size and shape of the airway.

10. The nasal insert of claim 9, wherein the adjustment system includes an expandable material.

11. The nasal insert of claim 1, wherein the nasal insert body includes one or more recesses or passages formed in a sidewall.

12. A nasal insert for modifying an airway of a person, the nasal insert comprising:
a body sized for insertion into a nasal passage of a person and formed from a bio- compatible material and comprising:
a corrective surface that is rigid or semi-rigid and configured to apply a corrective force to remodel an abnormality from the nasal passage during a treatment duration; and
an adjustment surface to facilitate insertion, removal, and adjustment of the body within the nasal passage to apply a desired pressure on the abnormality over the treatment duration.

13. The nasal insert of claim 12, wherein the adjustment surface includes a recess extending into the body and dimensioned to receive a digit of the person to facilitate self-deployment, self-adjustment, and self-removal.

14. The nasal insert of claim 12, wherein the adjustment surface includes a sloped surface integrally formed with the body.

15. A method of using a nasal insert to modify an airway to correct an abnormality, the method comprising:
identifying an abnormal airway;
inserting a first nasal insert selected from a series of nasal inserts into the airway to engage a nasal passage surrounding the airway, the first nasal insert having a first geometry;
removing the first nasal insert after a first period of time configured to permanently remodel the nasal passage by a first increment; and
inserting a second nasal insert selected from the series of nasal inserts into the nasal passage, the second nasal insert having a second geometry that is different from the first geometry and configured to permanently remodel the nasal passage by a second increment to correct the abnormality.

16. The method of claim 15, further comprising after removing the first nasal insert, adjusting the first nasal insert to form the second nasal insert.

17. The method of claim 16, wherein adjusting the first nasal insert includes changing an orientation of a first corrective surface of the first nasal insert.

18. The method of claim 15, wherein the series of nasal inserts includes at least five different nasal inserts.

19. The method of claim 15, wherein inserting the first nasal insert includes compressing a portion of the first nasal insert, moving the first nasal insert past a nostril opening, and expanding the first nasal insert within the nasal passage.

20. A method of producing a nasal insert for modifying an airway of a person, the method comprising:
acquiring shape information about a nasal passage of the person to identify a nasal abnormality in the airway of the person;
using the shape information, generating a nasal passage model;
using the nasal passage model, determining a treatment plan to modify the nasal passage of the person to correct the nasal abnormality; and
providing one or more nasal inserts having a geometry selected to implement the treatment plan to modify the nasal passage of the person to correct the nasal abnormality by deploying the one or more nasal inserts into the airway of the person.

21. The method of claim 20, wherein providing the one or more nasal inserts includes performing an additive manufacturing process to generate the one or more nasal inserts based on the nasal passage model.
